(19) United States
(12) Patent Application Publication
(10) Pub. No.: US 2020/0268966 A1
(43) Pub. Date: Aug. 27, 2020

(54) PROCEDURE TO OPERATE A PERFUSION DEVICE AND PERFUSION DEVICE TO IMPLEMENT THE PROCEDURE

(71) Applicant: ONEFUSION AG, Zurich (CH)

(72) Inventors: Daniel LÄNGLE, Zürich (CH); Dominic BÖNI, Zürich (CH)

(73) Assignee: onefusion AG, Zurich (CH)

(21) Appl. No.: 15/776,280

(22) PCT Filed: Nov. 11, 2016
(86) PCT No.: PCT/IB2016/056820

§ 371 Date: May 15, 2018

(30) Foreign Application Priority Data
Nov. 16, 2015 (AT) A 731/2015

(51) Int. Cl. A61M 5/168; A61M 5/14

(52) U.S. Cl. CPC A61M 5/1689; A61M 5/1411; A61M 5/1413; A61M 2205/3334; A61M 5/16881; A61M 2205/3368; A61M 5/16813

(57) ABSTRACT

Perfusion systems and procedures for operating perfusion systems that can include a drip container, a drop detector, a perfusion conduit and at least one control valve or a syringe pump. During the procedure, at least one calibration of the measuring process and the storage of at least one calibration value are performed. A control unit regulates the control valve or the syringe pump while using at least one measurement performed by the measuring device and taking into account at least one calibrated value towards the desired flow volume.

### CROSS REFERENCE TO RELATED APPLICATIONS

This application claims the benefit of Austrian Patent Application No. A 731/2015, filed Nov. 16, 2015. The disclosure of the prior application is considered part of and is incorporated by reference in the disclosure of this application.

### BACKGROUND

### 1. Technical Field

This disclosure relates to a procedure to operate a perfusion device comprising a drip container, a drop detector, a perfusion conduit and at least one regulating valve or one syringe pump and a perfusion conduit. The disclosure also relates to a perfusion device to implement this procedure.

### 2. Background Information

In customary perfusion devices, a container holding the perfusion liquid is positioned next to a drip container, to which is connected a perfusion conduit, specifically a perfusion tube. At the free end of the perfusion conduit is a perfusion needle, which is inserted into a vein of the patient who is to receive the perfusion. The perfusion conduit is fitted with a manually actuated control valve by which the flow velocity of the volume of perfusion liquid passing through the perfusion tube, and therefore the volume of perfusion liquid administered to the patient per time unit can be controlled.

During the use of such a perfusion device, the volume of perfusion liquid supplied to the patient per time unit is regulated by the control valve in the perfusion conduit, the objective being to meet the physiological needs of the patient. However, this procedure does not achieve the measurement of the flow velocity, nor is the course of the perfusion monitored. In a variation of this administration of a perfusion, a syringe pump is used instead of a perfusion container and a drip container. Here too, the flow velocity is not measured, nor is the course of the perfusion monitored.

### SUMMARY

This disclosure describes a procedure to operate a perfusion device comprising a drip container, a drop detector, a perfusion conduit and at least one regulating valve or one syringe pump and a perfusion conduit. The disclosure also describes a perfusion device to implement this procedure.

To be able to measure the volume of perfusion liquid administered to the patient per time unit, thereby permitting the flow velocity to be controlled and any changes to be detected in the flow of perfusion liquid through the perfusion conduit, e.g., changes due to blockages in the perfusion tube, this disclosure describes the use of a measuring device along (e.g., abutting) the perfusion conduit in order to measure the velocity of the perfusion liquid flowing through the perfusion conduit. The measured flow rate of the perfusion liquid can be shown on a display. This disclosure also describes a control unit to which the readings from the measuring device are sent, and by which the control/regulating valve is actuated to regulate the volume of perfusion liquid supplied to the patient per time unit.

In one aspect, this disclosure is related to a method for operating a perfusion device which comprises a drip container, a drop detector, a perfusion conduit and at least one control valve or one syringe pump. The method includes at least one calibration of a measuring process, and the storage of at least one calibrated value. The method being performed by means of the drop detector or the syringe pump and by means of at least one measuring device for measuring at least one volume of the perfusion liquid flowing through the perfusion conduit per time unit. The control valve or syringe pump is regulated during the perfusion by a control unit that uses at least one measurement performed by the measuring device, while taking into account at least one calibrated value towards the desired flow volume.

Such a method for operating a perfusion device may optionally include one or more of the following features. The method may be characterised by several successive calibrations being performed and the calibrated values being stored. The method may be characterized by the minimum number of one calibration being performed before or when the perfusion is started. The method may be characterized by calibrations also being performed during the perfusion. The method may be characterised by calibrations being performed throughout the entire duration of the perfusion. The method may be characterised by at least some of the calibrated values being used to regulate the perfusion. The method may be characterised by the measuring device for measuring at least one volume of liquid flowing through the perfusion conduit per time unit also being used as a drop detector. The method may be characterised by the use of the control unit to determine the occurrence of a malfunction during the perfusion and end the perfusion if necessary.

In another aspect, this disclosure is directed to a perfusion device for performing the aforementioned method. Such a perfusion device can include a drip container, a drop detector, a perfusion conduit and a control valve. The perfusion device can be characterised by the provision of a measuring device to measure at least one volume of the perfusion liquid flowing through the perfusion conduit per time unit as well as a control unit containing data storage. The outlets of the drop detector and the measuring device can be positioned next to the control unit that regulates the control valve and the volume of perfusion liquid flowing through the perfusion conduit.

In another aspect, this disclosure is directed to a perfusion device for performing the aforementioned method. Such a perfusion device can include a syringe pump (which may include a servomotor) and a perfusion conduit. The perfusion device can be characterised by the provision of a measuring device for measuring at least one volume of the perfusion liquid flowing through the perfusion conduit per time unit as well as a control unit containing data storage. The outlet of the measuring device and the control of the servomotor can be in communication with the control unit in order to regulate the volume of perfusion liquid flowing through the perfusion conduit.

The aforementioned perfusion devices may optionally include one or more of the following features. The perfusion devices may be characterised by the measuring device for measuring at least one volume of the perfusion liquid flow through the perfusion conduit per time unit being equipped with a sensor that is fixed to the perfusion conduit. The perfusion device may be characterised by the measuring device for measuring at least one volume of the perfusion liquid flow through the perfusion conduit per time unit being equipped with a sensor that forms a component separate from the perfusion conduit, where the perfusion conduit and the measuring device are made to abut one another to perform the measurement. The perfusion device may be characterised by the measuring device also performing the function of a drop detector. The perfusion device may be characterised by the sensor, which forms part of the measuring device for measuring at least one volume of the perfusion liquid flowing through the perfusion conduit, being provided with two temperature sensors positioned separately in the flow direction and a heating element between them.

In another aspect, this disclosure is directed to a system for measuring a flow rate of a perfusion liquid. The system includes: (i) a drop detector configured to detect drops of the perfusion liquid within a drip container; (ii) a flow rate sensor configured to abut against a perfusion conduit coupled to the drip container; and (iii) a control unit in communication with the drop detector and the flow rate sensor. The flow rate sensor is not fixed to the perfusion conduit. The flow rate sensor includes a first temperature sensor, a second temperature sensor, and a heating element between the first and second temperature sensors. The control unit is configured to determine the flow rate of the perfusion liquid using a difference in temperatures detected by the first and second temperature sensors.

Such a system for measuring a flow rate of a perfusion liquid may optionally include one or more of the following features. The control unit may be further configured to determine the flow rate of the perfusion liquid using a time difference between successive drops of the perfusion liquid within the drip container as detected by the drop detector and using a known volume of a drop of the perfusion liquid.

In another aspect, this disclosure is directed to a control device for controlling a flow rate of a perfusion liquid flowing through a perfusion conduit. The control device includes: a flow rate sensor configured to abut against the perfusion conduit; a regulating valve configured to adjustably squeeze the perfusion conduit to regulate the flow rate of the perfusion liquid flowing through the perfusion conduit; and a control unit in communication with the flow rate sensor and the regulating valve. The control unit is configured to determine the flow rate of the perfusion liquid using a difference in temperatures detected by the first and second temperature sensors and to adjust the control valve based on the determined flow rate of the perfusion liquid. The flow rate sensor is not fixed to the perfusion conduit. The flow rate sensor includes a first temperature sensor, a second temperature sensor, and a heating element between the first and second temperature sensors.

Such a control device may optionally include one or more of the following features. The flow rate sensor may abut against the perfusion conduit while the control device is arranged in a first configuration, and the flow rate sensor may be spaced apart from the perfusion conduit while the control device is arranged in a second configuration. A movable portion of the control device may be configured to couple with the perfusion conduit and to: (i) position the perfusion conduit in contact with the flow rate sensor in the first configuration and (ii) position the perfusion conduit separated away from the flow rate sensor in the second configuration. A portion of the regulating valve may be coupled to the movable portion of the control device. The control device may include an inlet end and an outlet end. While the perfusion conduit is coupled to the control device the perfusion liquid may flow through the perfusion conduit from the inlet end toward the outlet end. The flow rate sensor may be located closer to the inlet end than the regulating valve. The flow rate sensor may be configured to abut against a round outer wall of a standard tubing portion of the perfusion conduit.

In another aspect, this disclosure is directed to a system for measuring a flow rate of a perfusion liquid. The system includes a perfusion conduit fitted with a thin membrane portion and a flow rate sensor configured to abut against the thin membrane portion. The flow rate sensor is not fixed to the thin membrane portion. The flow rate sensor includes: a first temperature sensor; a second temperature sensor; and a heating element between the first and second temperature sensors.

Such a system to measuring a flow rate of a perfusion liquid may optionally include one or more of the following features. The flow rate sensor may be a component of a control device that has a first configuration and a second configuration. The flow rate sensor may abut against the perfusion conduit while the control device is arranged in the first configuration, and the flow rate sensor may be spaced apart from the perfusion conduit while the control device is arranged in the second configuration. The thin membrane portion may be biocompatible. The thin membrane portion may be generally planar. The first and second temperature sensors may be temperature-dependent resistors. The first and second temperature sensors may be thermopiles. The heating element may be an electrical resistance heater.

In another aspect, this disclosure is directed to a system for measuring a flow rate of a perfusion liquid. The system includes a flow rate sensor configured to abut against a perfusion conduit connected to a drip container and a control unit in communication with the flow rate sensor. The flow rate sensor is not fixed to the perfusion conduit. The flow rate sensor includes a first temperature sensor; a second temperature sensor; and a heating element between the first and second temperature sensors. Individual drops of perfusion liquid formed in the drip container are detectable by the flow rate sensor because temperatures detected by the first temperature sensor and the second temperature sensor shift as a result of perfusion liquid flow rate changes created by impacts from the individual drops of perfusion liquid. The control unit is configured to receive signals from the flow rate sensor corresponding to the detected individual drops of perfusion liquid and to determine a first flow rate of the perfusion liquid based on the signals. The control unit is further configured to use the first flow rate to calibrate the flow rate sensor such that flow rates detected by the flow rate sensor are based on the signals from the flow rate sensor corresponding to the detected individual drops of perfusion liquid.

Such a system for measuring a flow rate of a perfusion liquid may optionally include one or more of the following features. The control unit may be further configured to determine the first flow rate of the perfusion liquid based on a time difference between successive drops of the perfusion liquid within the drip container as detected by the flow rate sensor and using a known volume of a drop of the perfusion liquid.

In another aspect, this disclosure is directed to a perfusion conduit. The perfusion conduit includes a tube defining a lumen for conveying a perfusion liquid and an insert coupled with the tube. The insert defines a channel in fluid communication with the lumen such that perfusion liquid flowing through the lumen also flows through the channel. The insert includes a thin membrane overlaying the channel. The thin membrane is configured for abutting with a flow rate sensor having a heating element for increasing a temperature of the perfusion liquid flowing through the channel and one or more temperature sensors for measuring temperatures of perfusion liquid flowing through the channel at one or more regions within the channel.

Such a perfusion conduit may optionally include one or more of the following features. The thin membrane portion may be generally planar.

In another aspect, this disclosure is directed to a method of calibrating a system for measuring a flow rate of a perfusion liquid. The method includes the steps of: (i) receiving, by a control unit of the system, two or more signals, each signal of the two or more signals corresponding to an individual drop of the perfusion liquid formed within a drip container; (ii) determining, by the control unit of the system, a first flow rate of the perfusion liquid based on the two or more signals; and (iii) calibrating, by the control unit of the system, a flow rate sensor such that flow rates detected by the flow rate sensor are based on the two or more signals corresponding to the individual drops of perfusion liquid. Such a method of calibrating a system for measuring a flow rate of a perfusion liquid may optionally include one or more of the following features. The determining may be based on a time difference between sequential signals of the two or more signals. The determining may be based on a known drop size of the perfusion liquid. The two or more signals may be from a flow rate sensor that detects the individual drops of perfusion liquid. A first temperature sensor of the flow rate sensor and a second temperature sensor of the flow rate sensor may be used to detect a flow rate change of the perfusion liquid resulting from impacts of the individual drops of perfusion liquid. The two or more signals may be from a drop counter that detects the individual drops of perfusion liquid.

In another aspect, this disclosure is directed to a method of using a perfusion system. The method includes: (i) coupling a perfusion conduit to a control unit such that a flow rate sensor of the control unit abuts against the perfusion conduit but is not fixed to the perfusion conduit; (ii) detecting, using the flow rate sensor, a flow of perfusion liquid passing through the perfusion conduit; and (iii) regulating, using a regulating valve configured to adjustably occlude the perfusion conduit to regulate a flow rate of the perfusion liquid flowing through the perfusion conduit. The flow of perfusion liquid is responsive to the flow of perfusion liquid detected by the flow rate sensor.

Such a method of using a perfusion system may optionally include one or more of the following features. The flow rate sensor may abut against a round outer wall of a standard tubing portion of the perfusion conduit. The flow rate sensor may abut against a thin membrane wall of an insert coupled with the perfusion conduit. The method may also include calibrating, by the control unit, the flow rate sensor such that flow rates detected by the flow rate sensor are based on two or more signals from the flow rate sensor corresponding to individual drops of perfusion liquid. The calibrating may be based on a time difference between sequential signals of the two or more signals. The calibrating may be based on a known drop size of the perfusion liquid. The regulating may include regulating the flow of the perfusion liquid to a set point. The set point may be input to the control unit by a user.

In another aspect, this disclosure is directed to a method of using a syringe pump. The method includes detecting, using a flow rate sensor, a flow of perfusion liquid passing through a perfusion conduit as a result of pressure generated by a syringe; and regulating the pressure generated by the syringe responsive to the flow of perfusion liquid detected by the flow rate sensor.

Such a method of using a syringe pump may optionally include one or more of the following features. The method may also include calibrating, by a control unit of the syringe pump, the flow rate sensor such that flow rates detected by the flow rate sensor are based on a known flow rate generated by the syringe pump.

In another aspect, this disclosure is directed to a system for measuring a flow rate of a perfusion liquid. The system includes a drip container coupled to a perfusion conduit; a flow rate sensor configured to abut against the perfusion conduit; and a control unit in communication with the flow rate sensor. The flow rate sensor is not fixed to the perfusion conduit. The flow rate sensor includes a first temperature sensor; a second temperature sensor; and a heating element between the first and second temperature sensors. The control unit is configured to determine the flow rate of the perfusion liquid using a difference in temperatures detected by the first and second temperature sensors.

To perform an exact measurement of the volume of perfusion liquid flowing through the perfusion conduit per time unit, it is necessary to calibrate the measuring process, i.e., to detect the parameters that determine the measuring process in terms of the volume of perfusion liquid flowing through the perfusion conduit, and to store these parameters. For this purpose, every perfusion conduit, in particular every perfusion tube, should ideally be equipped with a storage device in which the calibration data for that particular perfusion tube are saved so that they are available to facilitate accurate measurements of the flow velocity when the particular perfusion conduit is being used. However, since perfusion conduits, especially perfusion tubes, are used only once for a perfusion and then disposed of, this would lead to insupportably high costs.

The systems and methods described herein therefore have an objective of creating an economically feasible procedure to operate a perfusion device that would enable a precise volume of perfusion liquid to be administered to a patient per time unit. As described further below, this is achieved by performing at least one calibration of the measuring process (e.g., drop detector or syringe pump) and by measuring at least one volume of the perfusion liquid flowing through the perfusion conduit per time unit, (where the control valve or syringe pump is regulated during the perfusion by using at least one measurement obtained by the measuring device), while taking into account at least one calibrated value for the intended flow volume.

It is preferable to perform several calibrations successively while storing the calibrated values. In so doing, at least one calibration can be performed before or when the perfusion begins. In addition, calibrations can also be performed during the perfusion or throughout the entire duration of the perfusion, with at least some of the calibrated values being used to regulate the perfusion.

Since the measuring devices described herein are extremely sensitive, such measuring devices can also advantageously be used as a drop detector to measure at least one volume of the perfusion liquid flowing through the infusion conduit per time unit.

If a malfunction occurs during the perfusion, it can advantageously be detected using the systems and methods described herein, and then displayed by the regulating unit. In addition, the perfusion can be stopped by the regulating unit if necessary.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described herein. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description herein. Other features, objects, and advantages of the invention will be apparent from the description and drawings, and from the claims.

### DESCRIPTION OF THE DRAWINGS

FIG. 1 is perspective view of a perfusion device with a perfusion conduit that is fitted with a regulating device to implement the procedure.

FIG. 2 is a perspective view of an initial implementation form of the regulating device in a larger scale than FIG. 1.

FIG. 3 is a perspective view of a second implementation form of the regulating device in a larger scale than FIG. 1.

FIG. 4 is a perspective view of a section of the perfusion conduit with an insert contained in it.

FIG. 5 is a side view of the section of the perfusion conduit with the insert according to FIG. 4.

FIG. 6 is a cross-sectional view of the insert along line IID-IID in FIG. 4.

FIG. 7 is a perspective view of the control device as in FIG. 2 with the cover plate removed.

FIG. 8 is a perspective view of a syringe pump.

FIG. 9 is a plot of a curve representing the output of a flow measurement sensor.

FIG. 10 is a schematic depiction of a flow measurement using a flow measurement sensor in accordance with some embodiments.

FIG. 11 is another plot of a curve representing the output of a flow measurement sensor with ΔT measurements taken as multiple differing flow rates.

FIG. 12 is a flowchart of a method for calibrating a flow measurement sensor using a drop counter system.

FIG. 13 is a flowchart of a method for calibrating a flow measurement sensor using a syringe pump.

Like reference numbers represent corresponding parts throughout.

### DETAILED DESCRIPTION

This disclosure describes procedures to operate perfusion devices that include components such as, but not limited to, a drip container, a drop detector, a perfusion conduit, a regulating valve, and/or a syringe pump. The disclosure also describes perfusion devices to implement the procedures.

One exemplary form, a perfusion device to perform the procedures described herein includes a drip container, a drop detector, a perfusion conduit and a regulating valve. The perfusion device also includes a measuring device to measure at least one volume of the perfusion liquid flowing through the perfusion conduit per time unit and a control unit containing data storage. Outputs from the drop detector and the measuring device are received by the regulating unit that controls the regulating valve to modulate the flow rate of perfusion liquid flowing through the perfusion conduit.

In another example form, a perfusion device to perform the procedures described herein includes a syringe pump which has a servomotor assigned to it and is fitted with a perfusion conduit. The perfusion device also includes a measuring device to measure at least one volumetric flow rate parameter of the perfusion liquid flowing through the perfusion conduit per time unit. The perfusion device also includes a regulating unit containing data storage. Outputs from the measuring device are used for the control of the servomotor by the regulating unit by which the volume of perfusion liquid flowing through the perfusion conduit is controlled.

In some embodiments, the measuring device that measures the flow rate of perfusion liquid flowing through the perfusion conduit can be equipped with a sensor that is fixed to the perfusion conduit. On the other hand, in some embodiments the measuring device is a sensor that is separate from the perfusion conduit. In such a case, the perfusion conduit and the measuring device can be reversibly brought together to abut each other in order to perform the measurement. Using this arrangement, the measuring device can be advantageously reused multiple times, in conjunction with multiple different perfusion conduits (which are disposed after a single use).

Furthermore, as described further below the measuring device can function as the drop detector instead of or in addition to the drop counter.

As described further below, the sensor device that forms a component of the volumetric flow rate measuring devices described herein can include two temperature sensors mounted separately along the flow direction, with a heating element situated between them.

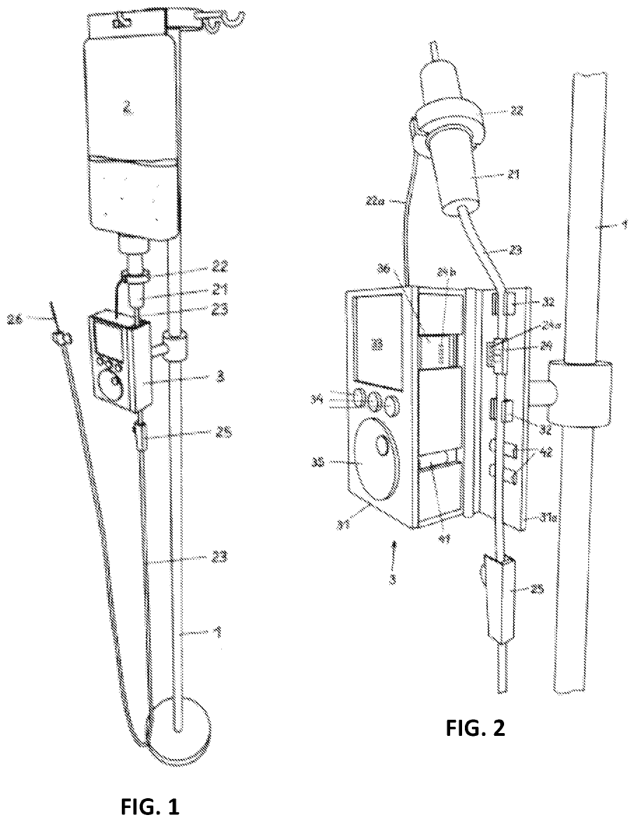

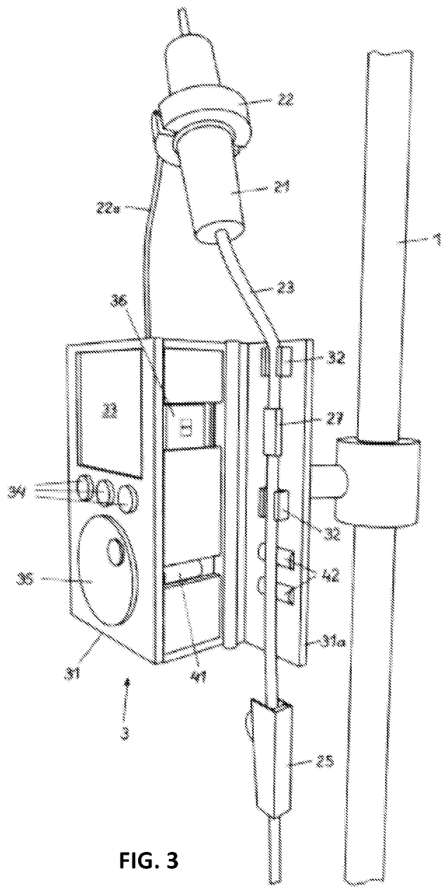

As can be seen in FIG. 1, a container 2 with a perfusion liquid is borne by a support frame 1. Connected to the container 2 is a drip container 21, which is fitted with a drop counter 22 as a drop detector. The drop counter 22 is fitted with a light barrier that counts the individual drops of perfusion liquid. The volume of the drops is known. Connected to the drip container 21 is a perfusion conduit 23 in the form of a perfusion tube, on which a roller clamp 25 is mounted. An injection needle 26 is fitted to the free end of the perfusion conduit 23. The perfusion conduit 23 is led through a control device 3, the layout and function of which are explained below with the aid of FIG. 2 and FIG. 3.

As shown by FIG. 2, the control device 3 has a housing 31 with a side wall 31a that is movable between an open configuration and a closed configuration (e.g., the side wall 31a pivots or folds open). When the side wall 31a is folded open, the perfusion conduit 23 can be inserted and be held in the housing 31 by clamps 32. The perfusion conduit 23 is provided with a sensor 24, by which the volumes of perfusion liquid flowing through the perfusion conduit per time unit can be determined. The sensor 24 is a component of a measuring device 36 that is contained in the housing 31, by which the volumes of perfusion liquid flowing through the perfusion conduit 23 per time unit are measured. User interface components such as a display 33, input buttons 34 and an adjusting wheel 35 are located on the front wall of the housing 31.

The control device 3 also contains a control slide valve 41 that can be positionally adjusted transversely to the perfusion conduit 23. The control device 3 has counter-bearings 42 coupled to it in the side wall 31a of the housing 31. When the side wall 31a is in the closed configuration, the sensor 24 is electronically connected via contact elements 24a and 24b with the measuring device 36.

The perfusion conduit 23 is then situated between the slide valve 41 and the counter-bearings 42 when the control device 3 is in the closed configuration. The slide valve 41 and the counter-bearings 42 corresponding to it form a squeeze valve in the perfusion conduit 23 (to adjustably occlude the lumen of the perfusion conduit 23), by which the volume of perfusion liquid flowing through it per time unit (i.e., flow rate of the perfusion liquid) can be adjustably controlled. The output of the drop counter 22 is attached to a control unit in the control device 3 by a line 22a.

The implementation form of the control device 3 portrayed in FIG. 3 differs from the implementation form shown in FIG. 2 in that the sensor for measuring the velocity of the perfusion liquid flowing through the perfusion conduit 23 is located directly inside the measuring device 36. In other words, the sensor is separate from the perfusion conduit 23. The perfusion conduit 23 is configured so that the measurements can be performed as soon as the perfusion conduit 23 is moved up against the measuring device 36 (e.g., when the control device 3 is in the closed configuration the sensor abuts the perfusion conduit 23). For this purpose, in the depicted embodiment the perfusion conduit 23 is fitted with an insert 27 that is explained below. In some embodiments, no such insert 27 is used and the sensor of the measuring device 36 can perform flow measurements by abutting against a normal tube portion (e.g., a standard, round outer wall of a tube) of the perfusion conduit 23.

According to the initial implementation form explained in reference to FIG. 2, the sensor 24 is fixed to the perfusion conduit 23 to determine the flow velocity. Since the perfusion conduit 23 is disposed of after every perfusion, the sensor 24 is disposed of too.

According to the second implementation form explained in reference to FIG. 3, the sensor to measure the flow velocity is not fixed to the perfusion conduit 23. Instead, the sensor is situated directly inside the measuring device 36, with the perfusion conduit 23 abutting on the sensor for measurements. This avoids the necessity of fitting the perfusion conduits 23 with sensors to detect the flow velocity. This achieves a major cost reduction because the sensors can be reused multiple times.

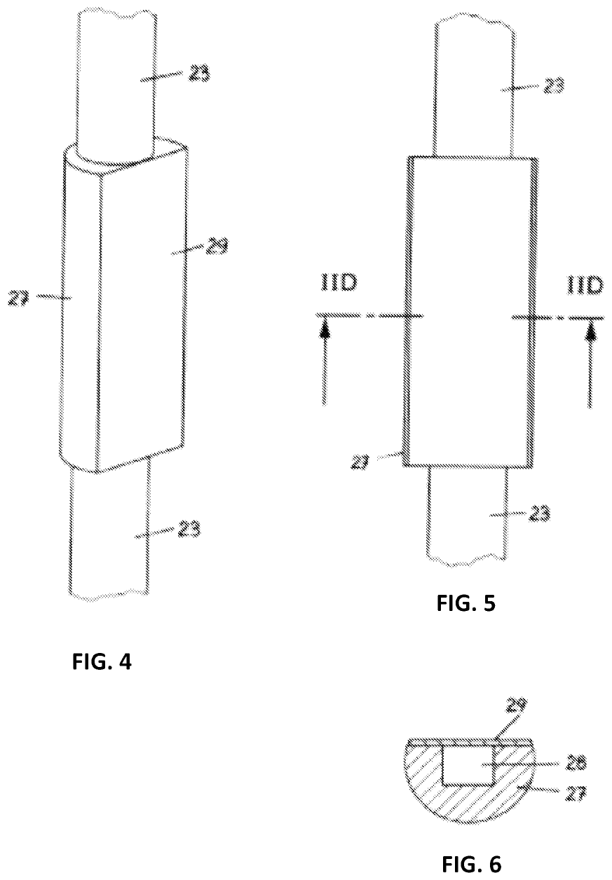

As made evident by FIG. 4, FIG. 5 and FIG. 6, a channel 28 is located in the insert 27 and is closed by a membrane 29. The membrane 29, which is manufactured from a plastic material, is extremely thin. While this membrane 29 abutting against (in contact with) the measuring device 36, the velocity of the perfusion liquid flowing through the perfusion conduit 23 is measureable by the measuring device 36.

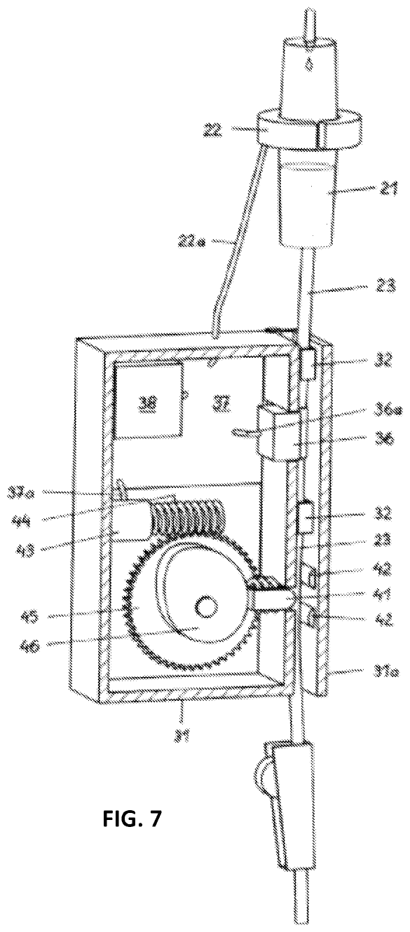

As is further evidenced by FIG. 7, in which the side wall 31a is moved (e.g., translated, pivoted, folded, etc.) into the position that closes the housing 31, the housing 31 contains the measuring device 36, the control unit 37 with data storage and a battery 38.

Housing 31 further contains a servomotor 43 with a worm gear 44, which meshes with a gear 45. The gear 45 has a cam 46, by which the control slide valve 41 can be shifted opposite the perfusion conduit 23. The outlet of the measuring device 36 is coupled with the control unit 37 via a conduit 36a. The control unit 37 is led via a conduit 37a to the servomotor 43 in order to adjust the slide valve 41.

In the initial state, the position of the control slide valve 41 is such that the perfusion conduit 23 is pinched closed by the squeeze valve formed by the control slide 41 and the counter-bearings 42. As soon as a perfusion is to be performed, an input button 34 is actuated for this purpose, causing the control slide valve 41 to be drawn back from the counter-bearings 42. This opens the squeeze valve, so that perfusion liquid will flow into the perfusion conduit from the drip container 21.

The flow of perfusion liquid that is consequently released by the drip container 21 is detected by the drop counter 22 which detects individual drops of the perfusion liquid. The signals output from the drop counter are transmitted via the line 22a to the control unit 37 and stored. The control unit 37, can determine the perfusion liquid flow rate based on the data received from the drop counter 22 and based on a known volume of a drop of the particular perfusion liquid being used.

In addition, the measuring device 36 measures the volume of perfusion liquid flowing through the perfusion conduit 23 per time unit. These measured values from the measuring device 36 are likewise transferred via the line 36a to the control unit 37 and stored. The control unit 37 can then use the flow rate determined from the drop counter 22 data to calibrate the measuring device 36. In other words, the flow rate detected by the measuring device 36 can be equated with the flow rate measured by the use of the drop counter 22. This accomplishes the calibration of the measuring process of the measuring device 36 that can be used to regulate the perfusion. The calibration takes into account variability in the parameters pertaining to the measuring process of the measuring device such as the perfusion conduit 23, the sensor 24 located on it or the sensor immediately contained inside the measuring device 36 and abutting the perfusion conduit 23, the spatial alignment of these components towards each other and similar factors.

The intended volumetric flow rate of perfusion liquid to be administered to the patient is entered as a set point via the user interface of the control device. Thereupon, using at least one measurement performed by the measuring device 36, and taking into account at least one calibrated value determined by means of the servomotor 43 via the worm gear 44, the gear 45 and the cam 46, the control slide valve 42 is adjusted to the setting that brings the squeeze valve formed in the perfusion conduit 23 by the control slide 41 and the counter-bearings 42 into the position required for the desired flow volume of perfusion liquid.

In addition, the control unit 37 can monitor for, detect and cause a notification (e.g., an alarm display and/or audible alarm) related to any malfunction (e.g., an excursion of the flow rate as measured in comparison to the flow rate set point), whereupon the perfusion is stopped if necessary. As soon as the perfusion is to be ended, the adjusting slide 41 moves the squeeze valve to the position that closes the perfusion conduit 23. Since the calibrated values are saved in the control unit 37 in both implementation variants, the perfusion conduit 23 does not require a storage unit.

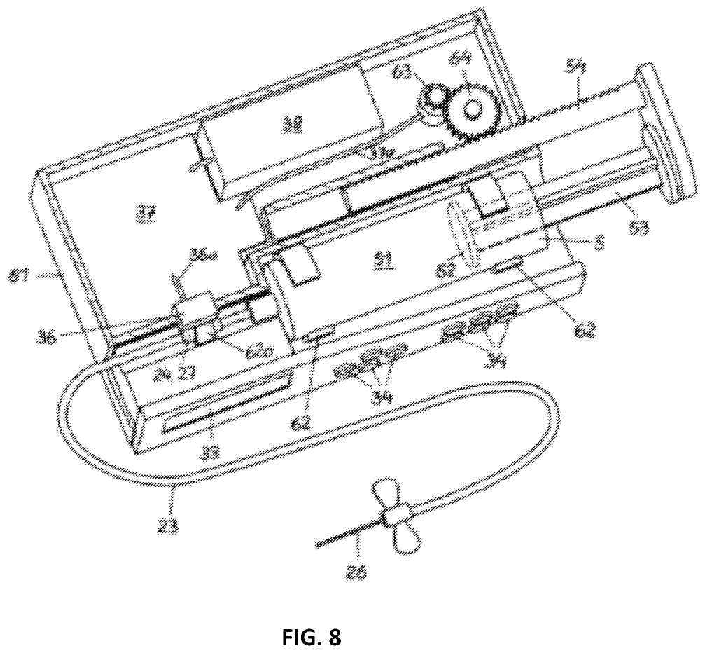

FIG. 8 depicts a syringe pump 5, which is inserted into a housing 61 and fastened inside it by clamps 62. The syringe pump 5 consists of a cylindrical container 51 holding a plunger 52 that can be adjusted by means of a plunger rod 53 projecting from the plunger 52 inside the container 51. The plunger rod 53 is fitted with a gear rod 54 that is positioned outside container 51. A perfusion conduit 23 in the form of a perfusion tube can be connected to the container 51 and has a perfusion needle 26 at its free end.

After perfusion liquid is drawn into the container 51 by adjusting the plunger 52, thereby filling the container 51, the latter is connected to the perfusion conduit 23 and the syringe pump 5 with the connecting section of the perfusion conduit 23, in which the sensor 24 or the insert 27 is located, is inserted in the housing 61 and held inside it by a clamp 62a.

The volumes of perfusion liquid flowing through the perfusion conduit 23 per time unit are determined by a sensor 24 in contact with the perfusion conduit 23. The sensor 24 is a component of a measuring device 36 located in the housing 61. The sensor 24 can be attached to the perfusion conduit 23 (e.g., as in FIG. 2) or separate from the perfusion conduit 23 (e.g., as in FIG. 3).

On the one hand, the sensor 24 can be fixed to the perfusion conduit 23 and be electronically connectable to the measuring device 36 via contact elements. Alternatively, the sensor 24 to measure the velocity of the perfusion liquid flowing through the perfusion conduit 23 can be located directly inside the measuring device 36, such that measurements can be performed while the perfusion conduit 23 is abutting against the measuring device 36.

The housing 61 further contains a servomotor 63, through which a pinion that meshes with the gear rod 54 can rotate. This enables the plunger 52 to be adjusted by the servomotor 63, the pinion 64, the gear rod 54 and the plunger rod 53, causing the perfusion liquid in the container 51 to be administered to the patient through the perfusion conduit 23 and the perfusion needle 26.

The housing 61 also contains the measuring device 36, a control unit 37 with a storage unit 37 and a battery 38. The output of the measuring device 36 is transmitted to the control unit 37 via a cable 36a. The control unit 37 is connected to the servomotor 63 via the control cable 37a. The housing 61 is also fitted with a display 33 and input buttons 34.

In some embodiments, the sensor 24 that determines the flow velocity is fixed to the perfusion conduit 23. Since the perfusion conduit 23 is disposed of after every perfusion, the sensor 24 is disposed of also. In some embodiments, the sensor 24 that measures the flow velocity is not fixed to the perfusion conduit 23. Instead, it is situated directly inside the measuring device 36, with the perfusion conduit 23 being made to abut the sensor 24 in order to detect the flow of perfusion liquid through the perfusion conduit 23. There is consequently no need to fit the perfusion conduits 23 with sensors to determine the flow velocity. This achieves a major cost reduction.

For this purpose, in some embodiments the perfusion conduit 23 is fitted with an insert 27 that is explained above in reference to FIGS. 3-6. While the membrane 29 is moved to abut (make contact with) the measuring device 36, the measuring device 26 measures the velocity of the perfusion liquid flowing through the perfusion conduit 23. In some embodiments, no insert 27 is used and instead the sensor 24 is moved to abut (make contact with) a standard portion of tubing of the perfusion conduit 23.

In the initial state, the plunger 52 is in the end position depicted in FIG. 8. When the servomotor 63 pressurizes the perfusion liquid within the container 51 by adjusting the plunger 52 in the cylindrical container 51, perfusion liquid flows through the perfusion conduit 23. Since the free cross-section of the cylindrical container 51 and the velocities v with which the plunger 52 is adjusted are known, the volume of perfusion liquid flowing through the perfusion conduit 23 per time unit is essentially known. On the other hand, the flow velocity in the perfusion conduit 23 is measured by the measuring device 36. The known volume of perfusion liquid flowing through the perfusion conduit 23 per time unit can be used to calibrate the measuring device 36. This enables at least one calibrated value to be determined before or at the beginning of the infusion, said calibrated value being subsequently taken into account in regulating the flow velocity of the perfusion liquid.

The desired volume of perfusion liquid to be administered to the patient is entered by an input button 34. The servomotor 63 is regulated by the control unit 37, using the measured value determined by the measuring device 36 and taking into account at least one calibrated value, to propel the perfusion liquid through the perfusion conduit 23 to the perfusion needle. In addition, the control unit 37 can monitor for, detect and cause a notification (e.g., an alarm display and/or audible alarm) related to any malfunction (e.g., an excursion of the flow rate as measured in comparison to the flow rate set point), whereupon the perfusion is stopped if necessary.

The perfusion procedure explained above consequently performs the calibration of the measuring process that is required for every perfusion conduit during a perfusion. This removes the need to calibrate perfusion conduits—particularly perfusion tubes with attached sensors—during their production and before their delivery and then store the calibration values in the storage elements located on the perfusion conduits, thereby achieving a major cost reduction.

The requirement of at least one calibration can be met before or at the start of the perfusion or while it takes place. Several successive calibrations can subsequently be performed and the calibrated values can be stored. In particular, calibrations can be performed throughout the entire duration of the perfusion. At least some of the calibrated values can be used to regulate the perfusion.

In addition to a single control valve, provision can be made for further control valves. Provision can likewise be made for adding further sensors to a single sensor.

Since the measuring device 36 (and sensor 24) described herein for measuring volume of the liquid flowing through the perfusion conduit per time unit is extremely sensitive, it is possible to use the measuring device 36 (and sensor 24) also as a drop detector as an alternative to, or in addition, to a drop counter with a light barrier. That is, the change in flow of the perfusion liquid related to an impact from a fallen drop of perfusion liquid (as formed in a drip container) is detectable by the measuring device 36 (and sensor 24).

A syringe pump is also understood to be an insulin pump, which can also be applied to perform this procedure.

The flow volume can be measured at various places along the perfusion conduit and also at the perfusion needle. Regarding the syringe pump in particular, the measurement can take place at the outlet of the cylindrical container 51.

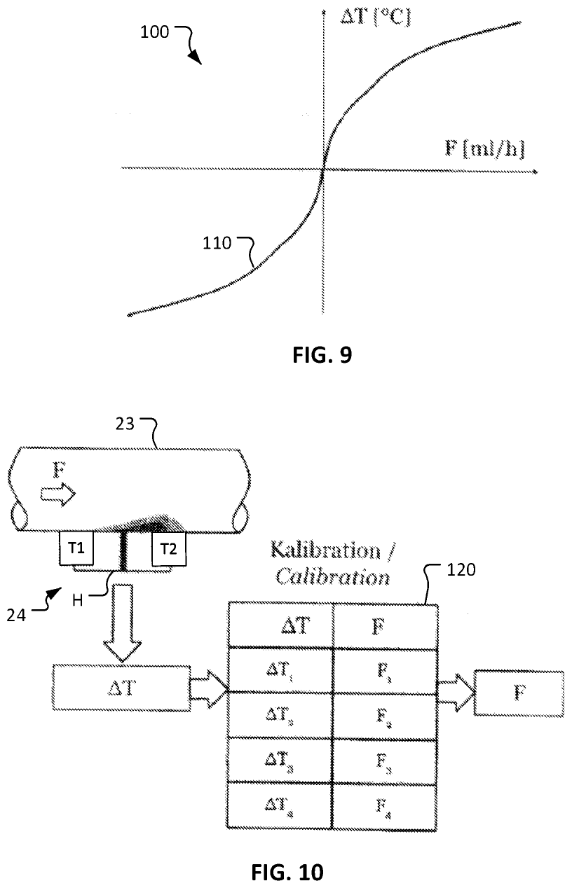

The calibration of the measuring procedure is explained further as follows. Referring to FIGS. 9 and 10, the sensor 24 is equipped with two temperature sensors T1 and T2, which are positioned separately in the flow direction of the perfusion liquid in the perfusion conduit 23, with a heating element H between them. The sensor 24 measures the flow rate F by means of detecting the temperature difference ΔT between the two sensor elements T1 and T2. As shown in FIG. 9, a graph 100 of flow rate versus ΔT includes a characteristic non-linear curve 110 which is known for this purpose.

This curve 110 is used as the basis for operating the sensor 24, and as one of the bases for calibrating the measuring process of the sensor 24. In practice, the actual shape of a particular characteristic curve 110 is effected by various parameters. Such parameters may include, but are not limited to, manufacturing tolerances of the perfusion conduit 23, the orientation of the sensor 24 in relation to the perfusion conduit 23, positions of the temperature sensors T1 and T2 and the heating element H towards each other etc. Therefore, the curve 110 (and, in turn, the sensor 24) can be calibrated using another reference (e.g., the known flow rates associated with a drop detector or syringe pump). For this calibration of the measuring process, at least one (and perhaps more than one) measurement is performed by means of which the temperature difference ΔT at a flow rate F is determined as illustrated in the table 120 of FIG. 10.

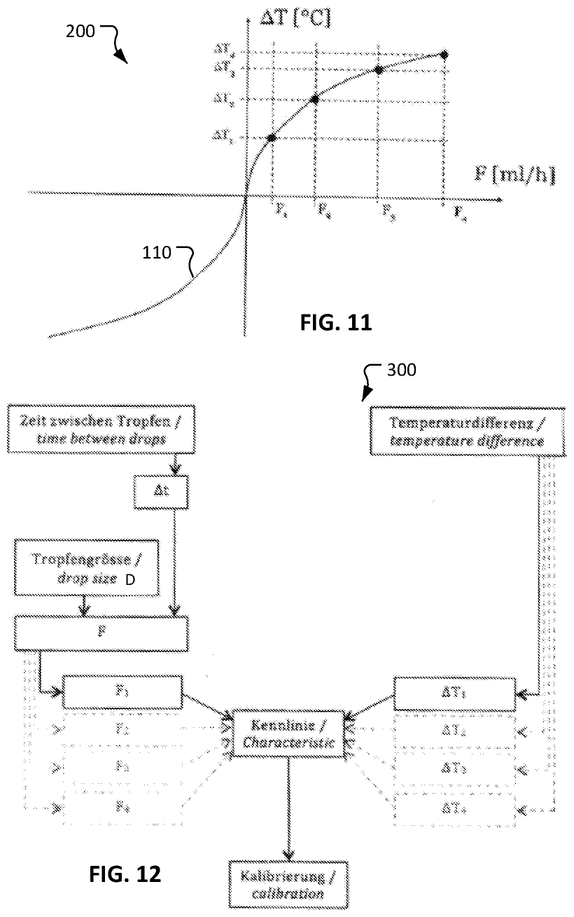

The characteristic curve 110 is adapted to the actual conditions by means of this measurement or these measurements. Intermediate values of the characteristic curve 110 are interpolated. As the number of measurements rises, the curve 110 becomes increasingly accurate as depicted in the graph 200 of FIG. 11.

This characteristic curve 110 is preferably charted in the area of the customary volumetric flow of liquid. The curve 110 is stored and taken into account in regulating the volume of perfusion liquid supplied to the patient per time unit.

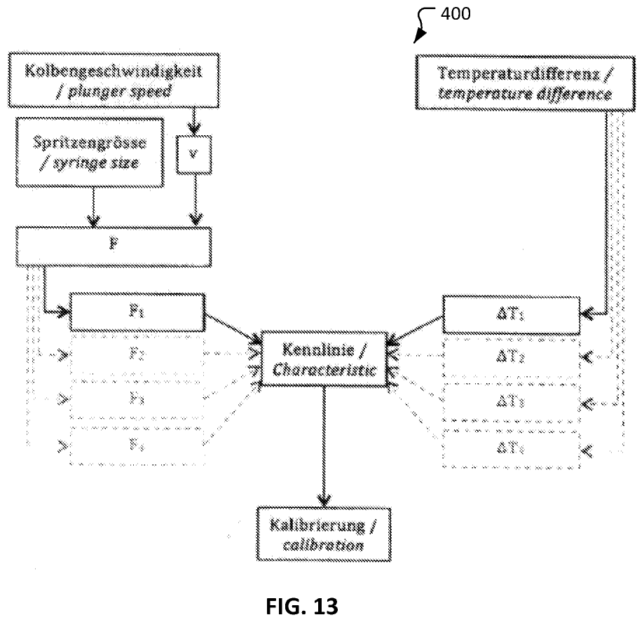

FIG. 12 and FIG. 13 schematically depict methods of calibrating the sensor 24 using a drop detector as a reference (FIG. 12) and using a syringe pump as a reference (FIG. 13), respectively.

As shown in the flowchart 300 of FIG. 12, the time between drops Δt (e.g., as measured by a drip counter or by the sensor 24) and the known volume of a drop D are combined to calculate a perfusion liquid flow rate F (e.g., F₁, F₂, F₃, F₄, and so on). Contemporaneously with the determination of F, a sensor 24 is operated and a temperature difference ΔT (e.g., ΔT₁, ΔT₂, ΔT₃, ΔT₄, and so on) is detected. F is then used as a calibration standard and ΔT is calibrated to agree with F.

As shown in the flowchart 400 of FIG. 14, the plunger speed v and the known syringe size are combined to calculate a perfusion liquid flow rate F (e.g., F₁, F₂, F₃, F₄, and so on). Contemporaneously with the determination of F, a sensor 24 is operated and a temperature difference ΔT (e.g., ΔT₁, ΔT₂, ΔT₃, ΔT₄, and so on) is detected. F is then used as a calibration standard and ΔT is calibrated to agree with F.

While this specification contains many specific implementation details, these should not be construed as limitations on the scope of any invention or of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described herein as acting in certain combinations and even initially claimed as such, one or more features from a claimed combination can in some cases be excised from the combination, and the claimed combination may be directed to a subcombination or variation of a subcombination.

It is very important to understand that one or more features from a particular device, system, or method described herein can be combined with one or more features from one or more other devices, systems, or methods described herein. Moreover, without limitation, all such combinations and permutations are within the scope of this disclosure.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described herein should not be understood as requiring such separation in all embodiments, and it should be understood that the described program components and systems can generally be integrated together in a single product or packaged into multiple products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In certain implementations, multitasking and parallel processing may be advantageous.

1.-14. (canceled)

15. A system for measuring a flow rate of a perfusion liquid, the system comprising:
a drop detector configured to detect drops of the perfusion liquid within a drip container;
a flow rate sensor configured to abut against a perfusion conduit coupled to the drip container, the flow rate sensor not fixed to the perfusion conduit, the flow rate sensor comprising:
a first temperature sensor;
a second temperature sensor; and
a heating element between the first and second temperature sensors; and
a control unit in communication with the drop detector and the flow rate sensor, the control unit configured to determine the flow rate of the perfusion liquid using a difference in temperatures detected by the first and second temperature sensors.

16. The system of claim 15, wherein the control unit is further configured to determine the flow rate of the perfusion liquid using a time difference between successive drops of the perfusion liquid within the drip container as detected by the drop detector and using a known volume of a drop of the perfusion liquid.

17. A control device for controlling a flow rate of a perfusion liquid flowing through a perfusion conduit, the control device comprising:
a flow rate sensor configured to abut against the perfusion conduit, the flow rate sensor not fixed to the perfusion conduit, the flow rate sensor comprising:
a first temperature sensor;
a second temperature sensor; and
a heating element between the first and second temperature sensors;
a regulating valve configured to adjustably squeeze the perfusion conduit to regulate the flow rate of the perfusion liquid flowing through the perfusion conduit; and
a control unit in communication with the flow rate sensor and the regulating valve, the control unit configured to determine the flow rate of the perfusion liquid using a difference in temperatures detected by the first and second temperature sensors and to adjust the control valve based on the determined flow rate of the perfusion liquid.

18. The control device of claim 17, wherein the flow rate sensor abuts against the perfusion conduit while the control device is arranged in a first configuration, and wherein the flow rate sensor is spaced apart from the perfusion conduit while the control device is arranged in a second configuration.

19. The control device of claim 18, wherein a movable portion of the control device is configured to couple with the perfusion conduit and to: (i) position the perfusion conduit in contact with the flow rate sensor in the first configuration and (ii) position the perfusion conduit separated away from the flow rate sensor in the second configuration.

20. The control device of claim 19, wherein a portion of the regulating valve is coupled to the movable portion of the control device.

21. The control device of claim 17, wherein the control device includes an inlet end and an outlet end, wherein while the perfusion conduit is coupled to the control device the perfusion liquid flows through the perfusion conduit from the inlet end toward the outlet end, and wherein the flow rate sensor is located closer to the inlet end than the regulating valve.

22. The control device of claim 17, wherein the flow rate sensor is configured to abut against a round outer wall of a standard tubing portion of the perfusion conduit.

23.-33. (canceled)

34. A method of calibrating a system for measuring a flow rate of a perfusion liquid, the method comprising:
receiving, by a control unit of the system, two or more signals, each signal of the two or more signals corresponding to an individual drop of the perfusion liquid formed within a drip container;
determining, by the control unit of the system, a first flow rate of the perfusion liquid based on the two or more signals; and
calibrating, by the control unit of the system, a flow rate sensor such that flow rates detected by the flow rate sensor are based on the two or more signals corresponding to the individual drops of perfusion liquid.

35. The method of claim 34, wherein the determining is based on a time difference between sequential signals of the two or more signals.

36. The method of claim 35, wherein the determining is based on a known drop size of the perfusion liquid.

37. The method of claim 34, wherein the two or more signals are from a flow rate sensor that detects the individual drops of perfusion liquid, and wherein a first temperature sensor of the flow rate sensor and a second temperature sensor of the flow rate sensor are used to detect a flow rate change of the perfusion liquid resulting from impacts of the individual drops of perfusion liquid.

38. The method of claim 34, wherein the two or more signals are from a drop counter that detects the individual drops of perfusion liquid.

39. (canceled)

40. The method of claim 37, wherein the flow rate sensor abuts against a round outer wall of a standard tubing portion of the perfusion conduit.

41. The method of claim 37, wherein the flow rate sensor abuts against a thin membrane wall of an insert coupled with the perfusion conduit.

42.-49. (canceled)
